(19) United States
(12) Patent Application Publication
(10) Pub. No.: US 2004/0171000 A1
(43) Pub. Date: Sep. 2, 2004

(54) Human est1 gene

(76) Inventors: Joachim Lingner, Epalinges (CH); Patrick Reichenbach, La Sarraz (CH); Matthias Hoss, Barnet (GB); Markus Nabholz, Etoy (CH); Philipp Bucher, Lausanne (CH)

Correspondence Address: NIXON & VANDERHYE, PC, 1100 N GLEBE ROAD, 8TH FLOOR, ARLINGTON, VA, 22201-4714, US

(21) Appl. No.: 10/473,115

(22) PCT Filed: Mar. 27, 2002
(86) PCT No.: PCT/GB02/01468

(30) Foreign Application Priority Data
Mar. 27, 2001 (GB) 0107627.2
Sep. 14, 2001 (GB) 0122304.9

(51) Int. Cl. C12Q 1/68; C07H 21/04; C12P 19/34; C12N 9/22

(52) U.S. Cl. 435/6; 435/69.1; 435/91.2; 435/199; 435/320.1; 435/325; 536/23.2

(57) ABSTRACT

The invention provides an isolated, enriched, purified or recombinant nucleic acid sequences (EST1) and proteins encoded thereby having activity as telomerase activity modulators. The invention also provides methods for screening for agents having mammalian telomerase modulating activity.

The present invention relates to novel proteins, peptide fragments and conjugates thereof, and nucleic acid encoding for the proteins and fragments, that have utility in modulation of telomere production. The novel protein, peptide fragments and DNA have utility in rendering cells capable of longer or shorter replicative life, at least in terms of number of replications before the cell exits the cell cycle, and in screening for agents that are capable of anticancer activity in so far as they modulate a cells ability to ‘immortalise’ itself.

Telomerase is a ribonucleoprotein particle required for the complete replication of chromosome ends (see Curr. Op. Genetics and Development 8, 226 (1998). The telomerase RNA moiety is tightly associated with telomerase proteins and serves as a template for telomerase repeat synthesis. Telomerase activity is absent in normal human somatic cells and therefore, chromosomal ends or telomeres shrink with each replication cycle in these cells (see Science 279, 349 (1998)). This deficit limits the replicative potential of normal human somatic cells. In 85% of all tumours telomerase is upregulated and telomerase activation appears to be a critical event during tumorigenesis (see Nature 400 464 (1999)). Telomerase inhibitors are of potential use for the treatment of neoplasias while the absence of telomerase activity in most normal tissues has been implicated in age-related diseases.

Yeast Est1p is a yeast telomerase component essential for the replication of telomeres. Upon deletion of the yeast EST1 gene, telomeres shrink with each replication cycle, as would be the case in a telomerase defect (see Cell 57, 633 (1989)). Est1p is associated with yeast telomerase RNA and binds to single-stranded telomerase DNA in vitro (see Current Biology 10, 809 (2000)). It is thought to mediate the interaction between the telomere and telomerase (Proc. Natl. Acad. Sci 94, 11190 (1997); Science 286, 117 (1999). However, human or vertebrate homologues of yeast Est1p have not been disclosed in the prior art. In fact the apparent absence of similar factors led to the hypothesis that vertebrates employ a different telomere replication mechanism. This hypothesis was supported by the discovery of large lariat structures called T-loops at human telomeres (see Griffith et al. Cell 1999 May 14;97(4):503-14).

The present inventors have identified human cDNA expressed sequence tag sequences that can be demonstrated to have low but significant sequence homology to yeast ESTI (see Table 1 and FIGS. 1 and 2 herein). Using reverse-transcriptase PCR (RT-PCR) they have amplified the cDNA sequences missing in deposited sequences and derived the complete open reading frames. Northern blot analysis (see FIG. 3 herein) demonstrates that these homologues are expressed in normal cells and in tumour derived cell lines (see FIG. 3).

The inventors have further provided antibodies against synthetic peptides predicted from the gene and protein sequences (see FIG. 4) and with one of these were able to immunoprecipitate the telomerase RNA moiety, demonstrating that this protein is associated with telomerase in vivo (see Table 2). Furthermore, they have demonstrated that the antibody specific for this protein also immunoprecipitates telomerase activity (see FIG. 5) which demonstrates that the protein is associated with active telomerase in vivo.

The inventors have still further demonstrated that, by expressing this protein in a human derived tumour cell line having telomerase activity, it is possible to interfere with native telomerase activity such that telomeres are reduced in length and thus their replicative capacity is reduced.

This is probably, but not exclusively, effected by recruitment of other telomerase obligate components away from the telomerase reverse transcriptase active subunit (hTERT or EST-2) by the overexpressed EST1. The EST1 protein may have to interact simultaneously with several ligands to fulfill its biological function. High levels of Est1 may lead to the formation of non-functional binary instead of functional ternary complexes. Possible ligands include hTERT or EST-2, telomerase RNA, telomeric DNA, Pot1 (cdc13) and other known and unknown telomere binding proteins. Over expressed levels of Est1 may lead to the formation of abnormally stable Est1-ligand complexes which prevent the ligand interacting with other molecules that act at the telomere. High levels of Est1 may induce a signal cascade that leads to repression (reduced expression or modification) of telomere replication factors.

Thus in a first aspect of the present invention there is provided an isolated, enriched, purified or recombinant nucleic acid sequence encoding for a protein having activity as a human telomerase activity modulator characterised in that it comprises a nucleotide sequence encoding for an amino acid sequence selected from those of SEQ ID No 2, SEQ ID No 4, SEQ ID No 5 and SEQ ID No 6 and sequences having at least 70% amino acid homology thereto and an active peptide fragment of any of these sequences.

More preferably the protein comprises an amino acid sequence of SEQ ID No 2 or 4 or one that has 75% or more homology, more preferably 80% or more and still more preferably 90% or more to one of those sequences.

More preferably the nucleic acid comprises a nucleic acid sequence selected from SEQ ID No 1 or SEQ ID No 3 or is capable of hybridising with nucleic acid having such a sequence under conditions of defined stringency.

Preferred nucleotide sequences include those that will hybridise with those of SEQ ID No 1 or 3 under standard stringency conditions of 2× SSC, more preferably high stringency conditions of 1× SSC (see Church and Gilbert, Proc Nat Acad Sci USA (1984) 81, 1991-1995 incorporated herein by reference). Preferred fragments, particularly of SEQ ID No 1 and homologues, are C-terminally truncated fragments.

It will be realised that by active fragments and homologues having the stated homology thereto we intend functional variants. A “functional variant” of a peptide or protein is a polypeptide the amino acid sequence of which can be derived from the amino acid sequence of the original peptide or protein by the substitution, deletion and/or addition of one or more amino acid residues in a way that, in spite of the change in the amino acid sequence, the functional variant retains at least a part of at least one of the biological activities of the original protein that is detectable for a person skilled in the art.

Preferably the amino acid sequence of the functional variant is at least 50% identical, more preferably at least 70% identical and most preferably at least 90% identical to the peptide or protein. Any functional part of a protein or a variant thereof is also termed functional variant. Biological function in the present application is the ability to modulate human telomerase activity in vivo or the activity of hTERT in vitro or in vivo.

By “homologous” is meant that the stated percentage of the amino acid sequence has identity or is of conservatively substituted amino acids. By “identical” is meant that the stated percentage of the amino acid sequence is identical. Both these percentage terms allow for gapping of sequences to allow alignment as is described below.

Particularly, by the term “identity” is meant that the stated percentage of the claimed amino acid sequence or base sequence is to be found in the reference sequence in the same relative positions when the sequences are optimally aligned, notwithstanding the fact that the sequences may have deletions or additions in certain positions requiring introduction of gaps to allow alignment of the highest percentage of amino acids or bases. Preferably the sequence are aligned by using 20 or less gaps, i.e. the total number of gaps introduced into the two sequences when added together is 20 or less, more preferably 10 or less. The length of such gaps is not of particular importance as long as the defined activity relating to modulation is maintained but generally will be no more than 50, and preferably no more than 10, amino acids, or 150 and preferably no more than 30 bases.

Variants from the aforesaid sequences preferably are conservative substitutions. The expression “conservative substitutions” as used with respect to amino acids relates to the substitution of a given amino acid by an amino acid having physicochemical characteristics in the same class. Thus where an amino acid has a hydrophobic characterising group, a conservative substitution replaces it by another amino acid also having a hydrophobic characterising group; other such classes are those where the characterising group is hydrophilic, cationic or anionic, or contains a thiol or thioether. Such substitutions are only contemplated where the resultant protein has hTERT modulating activity. For the present purposes proline may be treated as hydrophobic, but is preferably not substituted.

Algorithms and software suitable for use in aligning amino acid or nucleotide sequences for comparison and calculation of sequence homology or identity will be known to those skilled in the art. Significant examples of such tools are the Pearson and Lipman search based FAST and BLAST programs. Details of these may be found in Altschul et al (1990), J. Mol. Biol. 215: 403-10; Lipman D J and Pearson W R (1985) Science 227, p1435-41. Publicly available details of BLAST may be found on the internet at ‘http://www.ncbi.nlm.nih.gov/BLAST/blast-help.html’. Thus such homology and identity percentages can be ascertained using commercially or publicly available software packages incorporating, for example, FASTA and BLASTn software or by computer servers on the internet. Examples of the former are the GCG program package (Devereux et al Nucleic Acids Research (1984) 12 (1): 387) and the Bestfit program (Wisconsin Sequence Analysis Package, eg. Version 8 for Unix or IBM equivalent, Genetics Computer Group, University Research Park, 575 Science Drive, Madison, Wis. 53711), which uses the local homology algorithm of Smith and Waterman, Advances in Mathematics 2:482-489 (1981). Many international institutes, e.g. Genbank (see http://www.ncbi.nlm.nih.gov/BLAST) and EMBL (see http://www.embl-heidelberg.de/Blast2), offer internet services.

Parameters used in with software packages and internet servers should be applied with the appropriate sequence lengths and aforesaid gap characteristics in mind. Alignment strategies are discussed further in WO 98/40483 on pages 39 to 41, which document is incorporated herein by reference

Convenient parameters for BLAST searches are the default values, i.e. for EMBL Advanced Blast2: Blastp Matrix BLOSUMS 62, Filter default, Echofilter X, Expect 10, Cutoff default, Strand both, Descriptions 50, Alignments 50. For BLASTn defaults are again preferably used. GCG Wisconsin Package defaults are Gap Weight 12, Length weight 4. FASTDB parameters used for a further preferred method of homology calculation are mismatch penalty=1.00, gap penalty=1.00, gap size penalty=0.33 and joining penalty=30.0.

Nucleic acids of the invention may be degeneratively substituted with respect to that exemplified herein in the sequence listing. The expression ‘degeneratively substituted’ refers to substitutions of nucleotides by those which result in codons encoding for the same amino acid; such degenerative substitutions being advantageous where the cell or vector expressing the protein is of such different type to the DNA source organism cell that it has different codon preferences for transcription/translation to that of the cDNA source cell. Such degenerative substitutions will thus be host specific

Preferred encoded proteins may be provided as conjugated to other proteins, e.g. chromosome end capping proteins or peptides eg. such as cdc13, as fusion proteins and the nucleic acid, e.g. DNA, may encode for such fusions.

In a second aspect of the invention there is provided an isolated, enriched, purified or recombinantly produced protein comprising an amino acid sequence selected from the group consisting of SEQ ID No 2, SEQ ID No 4, SEQ ID No 5 and SEQ ID No 6 or a part of any of these, the protein or part thereof being effective to modulate the activity of telomerase in tumour cells in vivo or the ability of hTERT (human EST2) to extend telomere length in vitro.

It will be realised that one way to determine the ability of the protein to modulate the activity of telomerase in vivo will involve expressing nucleic acid, e.g. DNA, within the tumour cell and comparing the length of telomeres in that cell with that of telomeres in a cell of similar origin that lacks that expression of nucleic acid.

In a third aspect of the present invention there is provided antisense nucleic acid, particularly DNA, to that of the first aspect such that it is capable of downregulating expression of one or more of human EST1 gene products in vivo such that telomerase activity in a cell, particularly a tumour or precancerous cell, is reduced or eliminated. In this manner an ‘immortalised’ cancer or precancerous cell, e.g. a cell that would but for other anti-neoplastic agents, e.g. p53 or pRb, be cancerous, can be limited in replicative potential.

In a fourth aspect of the present invention there is provided a method for screening for agents having mammalian, and particularly human, telomerase modulating activity, in vivo or in vitro, comprising monitoring the interaction of a protein of the second aspect with one or more of the agents. It will be realised by those skilled in the art that such interaction will most conveniently be carried out in liquid medium, more preferably aqueous medium, typically of a physiologically compatible nature, eg. intracellular fluid mimicing assay buffer.

In a preferred embodiment of the fourth aspect of the present invention there is provided a method for screening for agents having mammalian, and particularly human, telomerase modulating activity, in vivo or in vitro, comprising monitoring the interaction of a protein of the second aspect with one or more of said agent(s), which comprises determining whether the presence of the agent modulates the interaction of the protein with one or more ligands selected from, mammalian DNA, mammalian telomerase RNA and a protein essential to the elongation of mammalian telomeres.

In a second preferred embodiment of the fourth aspect of the present invention there is provided a method for screening for agents suspected of having mammalian, particularly human, telomerase modulating activity, in vivo or in vitro, characterised in that it comprises (i) placing hTERT and a protein of the second aspect together in an aqueous medium together with DNA, telomerase RNA and the suspect agent, (ii) monitoring the ability of the hTERT (human EST2) to produce or extend telomeres on the DNA, (iii) determining any modulation of the elongation of telomeres in response to the agent as compared to a control medium having hTERT, protein, DNA, telomerase RNA but lacking the agent and (iv) designating the agent as a suspected inhibitor or activator of telomerase activity or as inactive accordingly.

It will be realised that the assay medium may further include a human end capping protein, such as cdc13 (Pot1) protein and any other cofactors that might be of utility in such a screening method. The method may also be carried out in a cell line lacking human telomerase activity.

In a third preferred embodiment of the fourth aspect of the present invention there is provided a method of screening for agents suspected of having mammalian, particularly human, in vivo or in vitro telomerase modulating activity characterised in that it comprises (i) providing one or more of human telomerase (hTERT or human EST2) and telomerase RNA together in an assay medium or cell together with a protein or fragment thereof of the second aspect and the suspected agent, (ii) lysing the cell if present and treating the lysate to provide a fraction containing the protein or fragment, (iii) mixing the fraction or medium containing the protein or fragment together binding agent specific for binding the protein or fragment and (iv) assessing the content of bound fragment from step (iii) for telomerase and/or telomerase RNA content and (v) correlating the amount of bound telomerase activity or RNA with activity of the suspect agent as either an inhibitor or activator of interaction of human in vivo telomerase activity or an inactive accordingly.

This aspect is conveniently carried out by use of a pull down assay with an antibody raised to the protein or fragment of the second aspect of the invention.

A further preferred embodiment of the fourth aspect is an immunoprecipitation assay wherein an intact telomerase complex (i.e. having all the components necessary for mammalian telomerase activity), in a form derived from a mammalian cell, is immunoprecipitated with an antibody generated against a protein of the second aspect or an immunogenic fragment thereof in the presence of an agent being screened for telomerase modulating activity; the presence of precipitated telomerase is detected and the amount thereof is correlated to the presence and amount of activity of modulating agent.

The telomerase is preferably in a form enriched from human cells, such as but not limited to tumour cells such as HeLa cells, and particularly from the mammalian cells nuclei.

A further preferred embodiment of the fourth aspect takes the form of a multi-hybrid binding assay using an organism, such as a yeast or a bacteria, to monitor the ability of a compound being screened to modulate the interaction of two or more of mammalian, preferably human, hTERT, Pot1 (cdc13) and hEST1 with a protein or fragment of the second aspect. It will be realised that hEST1 is a protein of the second aspect, but it has been determined by the present inventors that hEST1 is potentially capable of acting as a heterodimer or oligodimer, and thus any agent capable of modulating its interaction with itself can be calculated to have an effect on mammalian telomerase activity.

Preferred hybrid systems for use in this embodiment will combine two or three hybrid systems. Such systems commonly use yeast, but may use bacteria such as E. coli, for example as in the BacterioMatch system available from Stratagene wherein interacting molecules activate selectable ampicillin resistance gene and the lacZ gene. In this way the present inventors have been able to detect interactions between EST-1 molecules of the invention in the yeast two hybrid system indicating that it forms dimers or higher order multimers.

A further embodiment of the fourth aspect immobilises a protein or fragment of the second aspect onto a Scintillation Proximity Assay (SPA) material, such as a bead, exposes this to one or more of (a) a radiolabelled DNA oligo or polynucleotide corresponding to a mammalian hTERT sequence and (b) a radiolabelled telomerase RNA, such that the two bind whereby the labelled DNA is able to stimulate light emission from the beads, and correlates the difference in amount of light emitted from the beads in the presence of an agent to be screened and in its absence with the ability of the agent to modulate mammalian telomerase activity.

A further embodiment of the fourth aspect of the invention labels one of a protein of the second aspect of the invention or fragment thereof with a fluorescent label and labels one or more components selected from hTERT, telomerase RNA and mammalian DNA with a second fluorescent label, exposes these to each other in an aqueous medium, exposes the medium to light energy at a frequency at which one of the labels absorbs that energy, measures the amount of fluorescence emitted by the other label at a second frequency, relates the amount of that fluorescence to binding between the two labelled components, and correlates the difference in fluorescence in presence of an agent to be screened and in its absence with the ability of the agent to modulate telomerase activity.

Examples of labels are of green fluorescent protein (GFP) and blue fluorescent protein (BFP). When these proteins are of the order of 5 nanometers apart, such as requires direct interaction of the fusion protein, they transfer energy in this manner. Such a technique is known as two-fusion fluorescence energy transfer (FRET). Also provided is bioluminescent resonance energy transfer (BRET), wherein transfer of energy resulting from the degradation of coelenterazine by Renilla luciferase (Rluc) to green fluorescent protein or a red shift variant, enhanced yellow fluorescent protein (EYFP) causes emission of fluorescence. BRET is strictly dependent on the molecular proximity between the energy donor (Rluc) and acceptor (EYFP) makes it particularly appropriate for use in screening for modulators via modulation of that emission.

In a sixth aspect of the present invention there is provided an antibody capable of binding a protein or peptide of the second aspect in a preferred or specific manner. Such antibody may be monoclonal or polyclonal, immobilised or free, and is exemplified in the examples below as used in a pull down assay.

In a seventh aspect of the present invention there is provided a method of providing or enhancing human telomerase activity in a cell that lacks or has relatively low native human telomerase activity comprising recombinantly incorporating nucleic acid, particularly DNA, as claimed in the first aspect of the invention into the cell in functional relationship with a promoter active in said cell.

Preferably the cell is a human cell for which it is desired to increase, potentially indefinitely, the number of potential cell replication cycles. In this manner so called ‘immortalisation’ of cells of benefit can be provided. For example, human somatic cells of a type particularly suited to the production of a particular therapeutic or diagnostic peptide or protein may be enhanced with respect to replicative potential, thus rendering them more useful in large laboratory scale or industrial scale production of said peptide or protein.

It will be realised that recombinantly incorporating only nucleic acid, e.g. DNA, of the first aspect may not always be sufficient, and thus preferred methods will also include incorporation of one or more nucleic acids encoding human EST2 (hTERT), human EST3 and cdc13 or some other protein having telomere end capping function in humans. It may also involve expressing conjugations of the protein of the invention together with one or more of these, particularly a conjugate of the present hEST1 with human cdc13 or a protein having telomere end capping function in humans. Promoters active in a human cell will occur to those skilled in the art and include CMV, SV40 and mPSV promoters.

In an eighth aspect of the present invention there is provided a vector or recombinant DNA encoding a dominant negative mutant EST1 protein or fragment of the second aspect of the invention sufficient to inhibit telomerase subunit assembly or binding to telomeres when expressed in a cell, particularly a tumour cell.

In a ninth aspect of the present invention there is provided a method of treating a mammalian patient in need of therapy for a tumour which has telomerase activity comprising transforming the tumour cells with a vector comprising DNA of the eighth aspect. Preferred vectors may be in the form of a viral vector, such as those of adenovirus, lentivirus, adenovirus associated virus (AAV), parvovirus, herpes virus and other suitable gene therapy vectors. Particularly preferred are those viral vectors that include tumour or tissue specific promoters such as those referred to in PCT/US95/15455 or PCT/GB00/01142 incorporated herein by reference. Alternatively the transformation may be provided by integrating the nucleic acid, e.g. DNA, of the invention behind an existing promoter within a target cell.

In a tenth aspect of the present invention there is provided a method of treating a mammalian patient in need of therapy for a disease wherein cells are losing the ability to replicate with resultant deficit in tissue function characterised in that it comprises transforming one or more of these cells with nucleic acid of the seventh aspect such that it is in functional relationship with a promoter functional in said cells. Again this may be carried out using a vector such as a viral vector or may be carried out by directly integrating the DNA into the cell, and most advantageously may be integrated functionally with respect to an existing endogenous promoter. Alternatively a promoter can be inserted into an operatively functional position with respect to an existing, but otherwise inactive EST1 sequence.

In an eleventh aspect the present invention provides a vector, such as a viral vector described above in the ninth aspect, comprising a nucleic acid and optional promoter as described in the tenth aspect.

The method of the tenth aspect and vector of the eleventh aspect has particular application to diseases of ageing and wasting diseases.

A twelfth aspect of the present invention provides PCR primers comprising from 10 to 30 contiguous bases of the sequence SEQ ID No 1 or SEQ ID No 3 or complementary sequence thereto.

A thirteenth aspect of the present invention provides an oligonucleotide probe, having use in detecting nucleic acid of the invention, comprising from 15 to the full length contiguous sequence of SEQ ID No 1 or SEQ ID No 3. Preferably the probe is labelled, e.g. with radiolabeled phosphorous.

The present invention will now be described further by way of illustration only by reference to the following non-limiting examples, Figures and Sequence listing. Further embodiments of the invention falling within the scope of the claims will occur to those skilled in the art in the light of these.

Sequence Listing

SEQ ID No 1 is the nucleic acid sequence of a cDNA encoding a preferred protein of the invention, designated herein EST1-0732.

SEQ ID No 2 is the amino acid sequence of that preferred protein of the invention.

SEQ ID No 3 is the nucleic acid sequence of a cDNA encoding a second protein of the invention, designated herein EST1-1089.

SEQ ID No 4 is the amino acid sequence of that protein of the invention.

SEQ ID No 5 is the amino acid sequence of a further protein of the invention designated herein EST1-3.

SEQ ID No 6 is the amino acid sequence of a further protein of the invention designated herein EST1-4.

SEQ ID No 7 is the amino acid sequence of a peptide fragment used as an antigen to raise antibodies specific to the protein of SEQ ID No 2.

SEQ ID No 8 is the amino acid sequence of a peptide fragment used as an antigen to raise antibodies specific to the protein of SEQ ID No 4.

### FIGURES

FIG. 1 is an alignment of the amino acid sequences of SEQ ID No 2 (0732) and 4 (1089) with those of other EST1 proteins S. cerevisiae EST1 and YDR206W, S. plombe SPBC2D10-13 and SPBC2F12-03C, C. elegans SMG-7, D. melanogastar LD02558.

FIG. 2 is a Northern Blot for the DNA of SEQ ID No 3.

FIG. 3 shows a telomerase assay of immunoprecipitates produced using antibodies to the proteins of SEQ ID No 2 (0732) and 4 (1089).

FIG. 4 shows diagrammatic representation of the constructs used in HeLa cell studies of Example 5.

FIG. 5 shows the effect of these studies on HeLa cell telomere length.

FIG. 6 shows the telomerase activity of affinity-purified telomerase fractions immunoprecipitated from HeLa NXT cells using αhEst1-antibodies raised to 0732 immunogenic peptide by the method of the invention as expressed on gels.

FIG. 7 shows the total activity of affinity purified telomerase fractions immunoprecipitated from HeLa NXT cells using using αhEst1-antibodies raised to 0732 immunogenic peptide (SEQ ID No 7) by the method of the invention.

FIG. 8 shows a Northern Blot Analysis of HEST Expression showing that this occurs in a complete variety of human tissues.

### EXAMPLES

### Example 1

Production of cDNAs encoding SEQ ID No 2 and 4.

DNA was produced using 5′RACE technique using human genomic RNA as template and suitable primers targeted at the ends of the sequences shown in the sequence listing. Similar techniques were used to obtain the sequences encoding SEQ ID No 5 and 6.

### Example 2

Expression of Human EST1 Proteins of SEQ ID No 2 and 4.

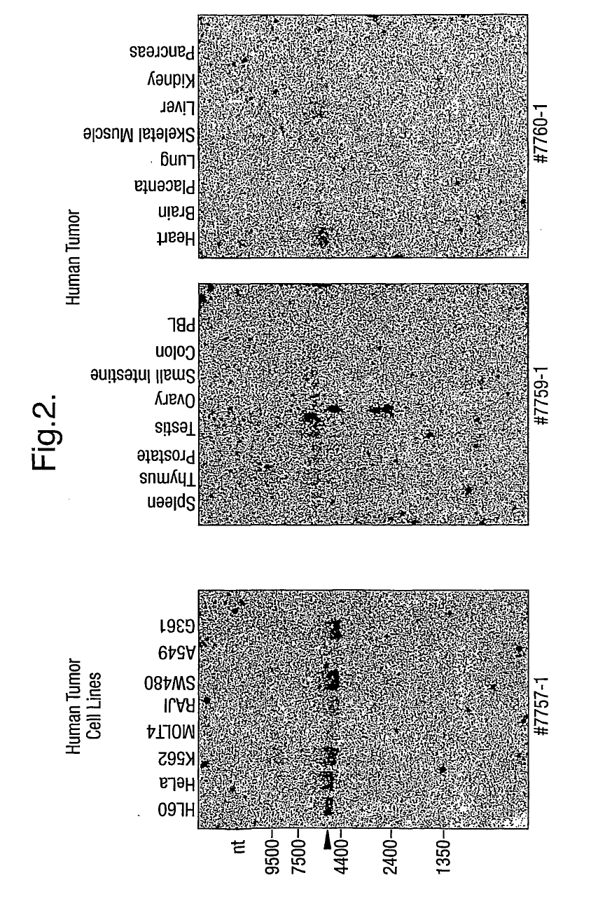

Northern blots were produced from RNA fractions derived from a variety of different human tissues and probed using radiolabeled oligonucleotide probes specific to DNA of SEQ ID No 1 and SEQ ID No 3. FIG. 2 shows the distribution of the mRNA encoding SEQ ID No 3 while similar distribution was found for SEQ ID No 1 (not shown).

### Example 3

Production of Antibodies to Proteins Comprising SEQ ID No 2 and SEQ ID No 4.

Peptides of 40 amino acids length were designed for each of the four homologues SEQ ID No 2, 4, 5 and 6. 500 μg of each peptide were used to immunise two rabbits per peptide, following the Eurogentec Bel S. A. (Herstal, Belgium) standard protocol. Intradermic multisite injections of 100 μg peptide (500 μl antigen solution, 500 μl adjuvant) were performed on days 0, 14, 28 and 56. Test blood samples (days 38 and 66) were checked for affinity to the peptides. All antibodies were affinity purified on antigen columns as described in Sambrook, J., Fitsch, E. F. and Maniatis, T. (1989). Molecular cloning, a laboratory manual, Cold Spring Harbor, N.Y. (incorporated herein by reference). The peptide an antibody was raised against was coupled to NHS-activated Sepharose 4 Fast Flow (Pharmacia) and washed extensively at low and high pH. Blood samples (20 ml) of immunized rabbits were passed over these antigen columns, the columns were washed, and bound antibodies eluted at high and low pH. Elution fractions were combined, concentrated and tested for antigen affinity in western blots.

### Example 4

Telomerase Assay (TRAP) of Immunodrecipitates.

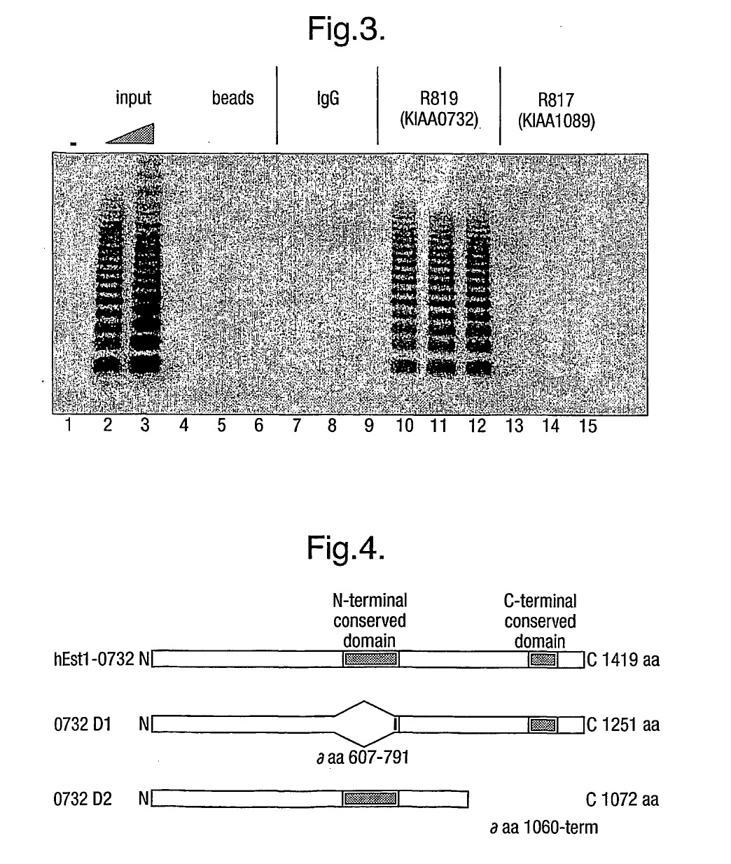

As shown in FIG. 3, HeLa cell nuclear extract (Lanes 1,2) was incubated with Protein A sepharose beads in the absence of antibody (lanes 4-6), presence of antibody to protein of SEQ ID No 7 (lanes 10-12) and antibody to protein of SEQ ID No 8 (lanes 13-15) and IgG negative control (lanes 7-9). The experiment was done in triplicate. Telomerase activity was specifically immunoprecipitated with antibody to SEQ ID No 7 (and thus SEQ ID No 2) demonstrating the association of 0732 (SEQ ID No 2) with active telomerase.

### Example 5

Telomerase Assay of Immunoprecipitates.

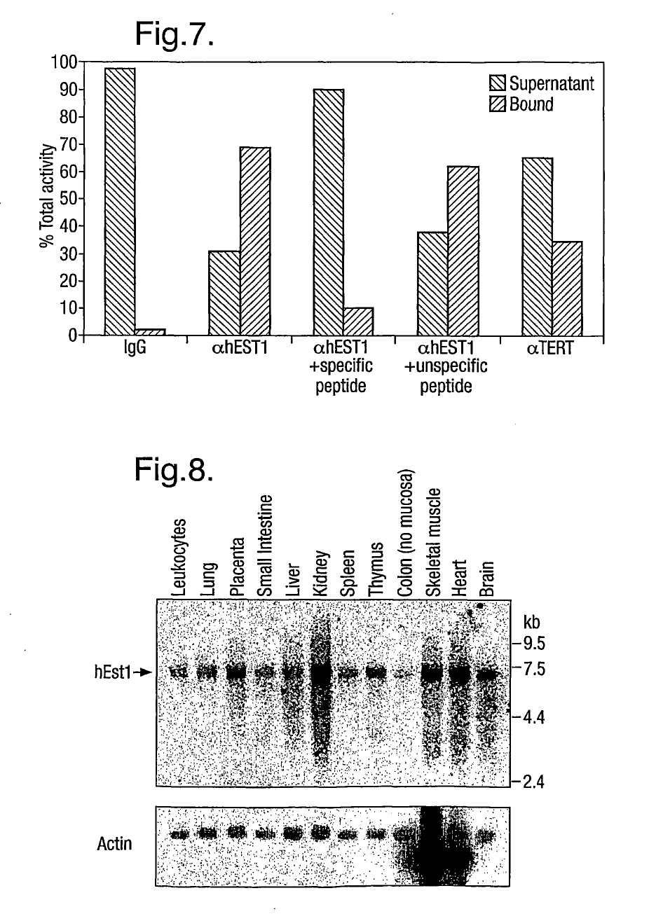

Immunoprecipitations were performed as described in Example 4 using a human telomerase fraction that had been affinity purified approximately 200-fold from HeLa cell nuclear extracts (see Schnapp et al. Nucleic Acids Res. 1998 Jul. 1;26(13):3311-3) FIG. 7 shows the results of the immunoprecipitation. Immunoprecipitation using antibodies to SEQ ID No 7 precipitated 70% of total telomerase Antibodies against human telomerase hTERT, which served as a positive control precipitated 35% of the activity, whereas an unspecific rabbit IgG failed to immunoprecipitate significant amounts of telomerase activity. Immunoprecipitation of telomerase activity by antibodies against SEQ ID No. 7 was completely abolished by addition of a 3.5 molar excess of the peptide against the antibody was raised, whereas a nonspecific peptide did not compete. These results indicate that most or all active telomerase is associated with a protein that contains the antigen, most likely the EST-1 protein containing the peptide SEQ ID No. 7.

### Example 6

Expression of Est1-0732 Constructs in HeLa Cells—Effects on Telomere Length.

Various Est1-0732 (SEQ ID No 2) constructs were overexpressed in HeLa cells I order to determine whether they had any in vivo activity with respect to telomere length.

Constructs used were as follows:

pCMV0732: Full length open reading frame of Estl-0732 in pEGFP-C2 (Clontech) from which GFP was removed by cutting the pEGFP-C2 vector with NheI and SmaI. The promoter used to drive over-expression was the CMV promoter.

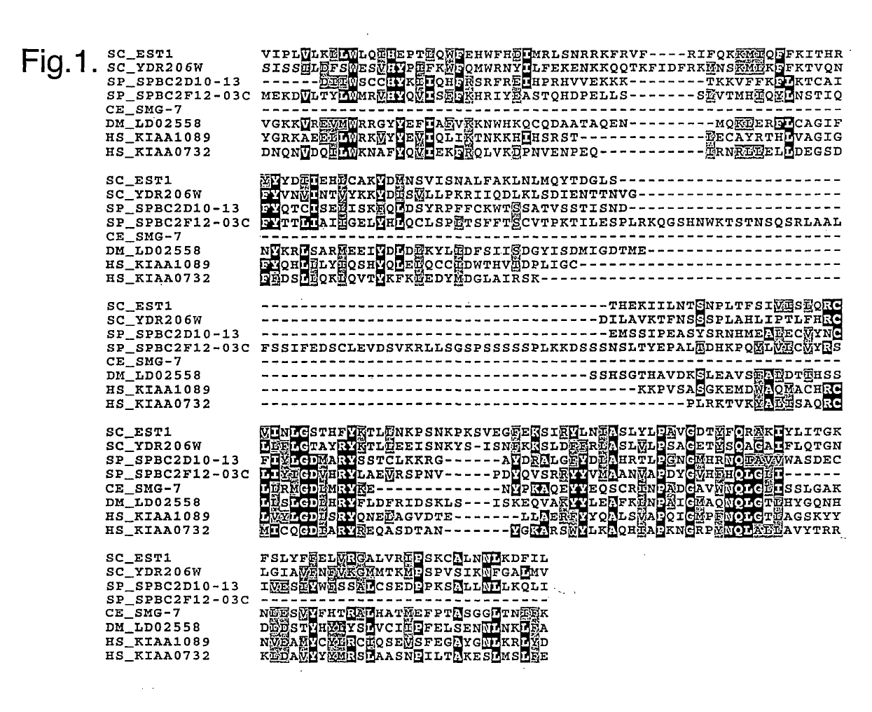

0732 D1: pCMV0732 was cut with Acc III and Fsp I, filled in to blunt end with Klenow enzyme in the presence of dNTPs and religated. This leads to a deletion of amino acids 607-791, which correspond to the region that is conserved among the Est1-family members shown in the alignment of FIG. 1.

0732 D2: pCMV0732 was cut with Sal I and religated. This leads to deletion of amino acids 1060-end.

Transfection of HeLa Cells:

HeLa cell were transfected using the GenePORTER transfection reagent (from AXON LAB AG). Cells were selected on neomycin and grown under standard conditions.

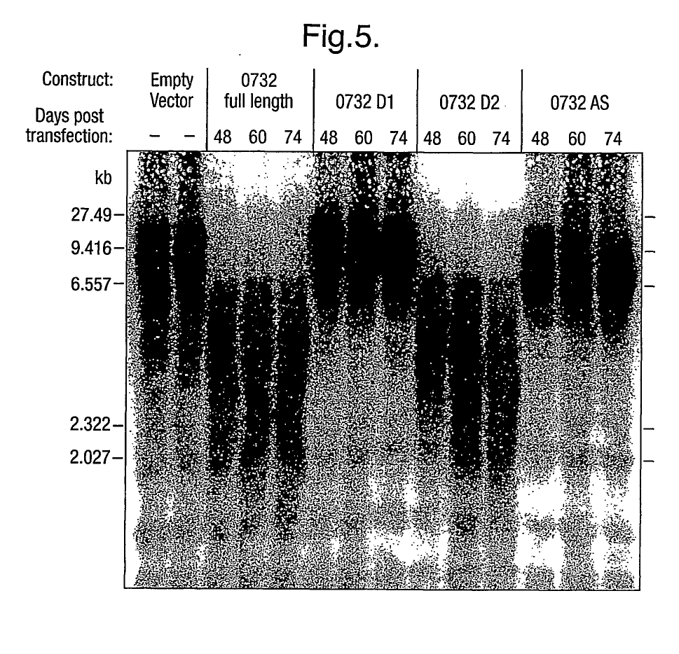

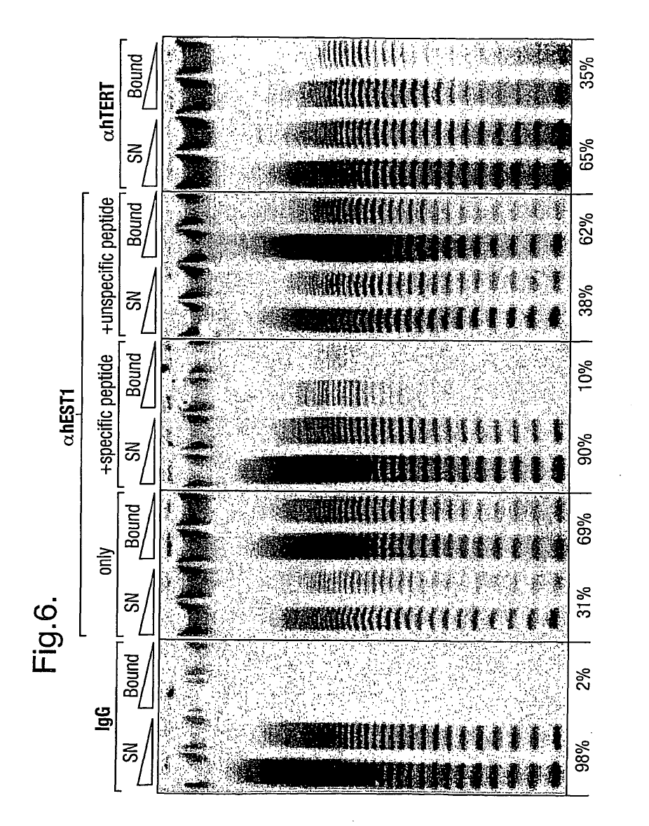

FIG. 4 shows diagrammatic representation of these constructs while FIG. 5 shows the effect of transfecting the cells on telomere length, with the empty vector, D1 and AS having little or no effect but full length SEQ ID No 1 and D2 vectors showing clear reduction in telomere length, providing evidence that SEQ ID No 1 and C-terminal truncates thereof have activity in recruiting components of the telomerase obligate components away from hTERT.

### Example 7

Multiple Tissue Northern (MTN)

A multiple tissue Northern blot was carried out using kit from Clontech (catalog No. 7780-1). The blot was hybridised with a probe made from a full length cDNA fragment of hEst1 that was labelled by random labelling with short oligonucleotides hexamers using standard protocols (Sambrook et al. Molecular cloning, CSH). The blot was also hybridised for control purposes with a human beta-actin cDNA probe provided by Clontech. FIG. 8 shows that hEST1 is expressed in a variety of human tissues.

### Example 8

Simple Screening Assay for EST1 Interactors.

Activity in binding to hEST1 protein can be screened, at an initial level a least, by combining candidate interacting agents, in aqueous physiological buffer solution, with that protein. Both agent and protein are initial solubilised and, after combining and a suitable time to allow interaction, the protein is precipitated by salting, as is well known in the art. The amount of agent remaining in solution is measured, eg. by OD value, and inversely related to its ability to bind the hEST1 protein and thus its suitability for further screening is assessed.

1. An isolated, enriched, purified or recombinant nucleic acid sequence encoding for a protein having activity as a human telomerase activity modulator characterised in that it comprises a nucleotide sequence encoding for an amino acid sequence selected from those of SEQ ID No 2, SEQ ID No 4, SEQ ID No 5 and SEQ ID No 6 and sequences having at least 70% amino acid homology thereto and an active peptide fragment of any of these sequences.

2. A nucleic acid as claimed in claim 1 encoding a protein comprising an amino acid sequence of SEQ ID No 2 or 4 or one that has 75% or more homology, more preferably 80% or more and still more preferably 90% or more to one of those sequences.

3. A nucleic acid as claimed in claim 1 or 2 comprising a nucleic acid sequence selected from SEQ ID No 1 or SEQ ID No 3 or which is capable of hybridising with nucleic acid having such a sequence under conditions of standard or high stringency.

4. A nucleic acid as claimed in any one of the preceding claims encoding for a fragment of said protein characterised in that it is a C-terminally truncated fragment of said protein.

5. A nucleic acid as claimed in any one of the preceding claims characterised in that it encodes for a protein as described in any one of the preceding claims conjugated to other proteins, eg chromosome end capping proteins or peptides eg. such as cdc13, as fusion proteins.

6. An isolated, enriched, purified or recombinantly produced protein comprising an amino acid sequence selected from the group consisting of SEQ ID No 2, SEQ ID No 4, SEQ ID no 5 and SEQ ID No 6 or a part of any of these, the protein or part thereof being effective to modulate the activity of telomerase in tumour cells in vivo or the ability of hTERT (human EST2) to extend telomere length in vitro.

7. A protein or part as claimed in claim 6 as encoded by any one of the nucleic acids of claims 1 to 5.

8. Antisense nucleic acid to nucleic acid as claimed in any one of claims 1 to 5.

9. Antisense nucleic acid as claimed in claim 8 characterised in that it is capable of downregulating expression of one or more of human EST1 gene products in vivo such that telomerase activity in a cell in which it is provided, particularly a tumour or precancerous cell, is reduced or eliminated.

10. A method for screening for agents having mammalian telomerase modulating activity, in vivo or in vitro, comprising monitoring the interaction of a protein as claimed in claim 6 with one or more of said agents.

11. A method for screening for agents having mammalian telomerase modulating activity, in vivo or in vitro, as claimed in claim 10, comprising determining whether the presence of one or more of said agent(s) modulates the interaction of the protein with one or more ligands selected from mammalian DNA, mammalian telomerase RNA and a protein essential to the elongation of mammalian telomeres.

12. A method for screening for agents having mammalian telomerase modulating activity, in vivo or in vitro, as claimed in claim 10 characterised in that it comprises (i) placing hTERT and a protein of the second aspect together in an aqueous medium together with DNA, telomerase RNA and the suspect agent, (ii) monitoring the ability of the hTERT (human EST2) to produce or extend telomeres on the DNA, (iii) determining any modulation of the elongation of telomeres in response to the agent as compared to a control medium having hTERT, protein, DNA, telomerase RNA but lacking the agent and (iv) designating the agent as a suspected inhibitor or activator of telomerase activity or as inactive accordingly.

13. A method of screening for agents suspected of having mammalian, in vivo or in vitro, telomerase modulating activity as claimed in claim 10 characterised in that it comprises (i) providing one or more of human telomerase (hTERT or human EST2) and telomerase RNA together in an assay medium or cell together with a protein or fragment thereof of the second aspect and the suspected agent, (ii) lysing the cell if present and treating the lysate to provide a fraction containing the protein or fragment, (iii) mixing the fraction or medium containing the protein or fragment together binding agent specific for binding the protein or fragment and (iv) assessing the content of bound fragment from step (iii) for telomerase and/or telomerase RNA content and (v) correlating the amount of bound telomerase activity or RNA with activity of the suspect agent as either an inhibitor or activator of interaction of human in vivo telomerase activity or an inactive accordingly.

14. An antibody capable of binding a protein or peptide as claimed in claim 6 or claim 7.

15. A method of providing or enhancing human telomerase activity in a cell that lacks or has relatively low native human telomerase activity comprising recombinantly incorporating nucleic acid, particularly DNA, as claimed any one of claims 1 to 5 into the cell in functional relationship with a promoter active in said cell.

16. A vector or recombinant DNA encoding a dominant negative mutant EST1 protein or fragment as claimed in claim 6 or 7 sufficient to inhibit telomerase subunit assembly or binding to teleomeres when expressed in a cell, particularly a tumour cell.

17. A method of treating a mammalian patient in need of therapy for a tumour which has telomerase activity comprising transforming the tumour cells with a vector comprising DNA as claimed in claim 16.

18. A method of treating a mammalian patient in need of therapy for a disease wherein cells are losing the ability to replicate with resultant deficit in tissue function characterised in that it comprises transforming one or more of these cells with nucleic acid as claimed in any one of claims 1 to 5 such that it is in functional relationship with a promoter functional in said cells.

19. A vector, such as a viral vector comprising a nucleic acid and optional promoter as claimed in any one of claims 1 to 5.

20. A PCR primers comprising from 10 to 30 contiguous bases of the sequence SEQ ID No 1 or SEQ ID No 3 or complementary sequence thereto.

21. An oligonucleotide probe comprising from 15 to the full length contiguous sequence of SEQ ID No 1 or SEQ ID No 3 or complementary sequence thereto.

22. An assay composition comprising a DNA, cell, antibody or protein as claimed in any one of claims 1 to 21 together with one or more of mammalian teleomerase, telomerase RNA and a DNA to be extended.

23. An assay apparatus capable of screening for agents having mammalian telomerase modulating activity characterised in that it comprises a DNA, protein, cell or antibody as claimed in any one of claims 1 to 22.

24. A composition for modulating mammalian telomerase activity characterised in that it comprises at least one agent that has been identified as a modulator of said mammalian telomerase activity by a method as claimed in any one of claims 10 to 13.

25. A composition for modulating mammalian telomerase activity characterised in that it comprises as least one agent that has been identified as a modulator of said mammalian telomerase activity using a DNA, protein, cell or antibody as claimed in any one of claims 1 to 24.

26. A method of treating a patient in need of therapy for a disease involving abnormal replication of cells comprising administering to the patient an effective amount of an active as identified by a method as claimed in any one of claims 10 to 13.

27. A method of treating a patient in need of therapy for a disease involving abnormal replication of cells comprising administering to the patient an effective amount of an active as identified using a DNA, protein, cell or antibody as claimed in any one of claims 1 to 24.

28. Use of an agent that has been identified as a modulator of said mammalian telomerase activity by a method as claimed in any one of claims 10 to 13 for the manufacture of a medicament for modulating mammalian telomerase activity in a telomerase associated disease.

29. Use of an agent that has been identified as a modulator of said mammalian telomerase activity using a DNA, protein, cell or antibody as claimed in any one of claims 1 to 24 for the manufacture of a medicament for modulating mammalian telomerase activity in a telomerase associated disease.


SEQUENCE LISTING (14 sequences)

SEQ ID NO: 1 - DNA, 4260 bp, Homo sapiens
  CDS (1)..(4260)
atggcggaagggctggagcgtgtgcggatctccgcgtcggagctgcgcgggatcctggctactctggccccgcaggccgggagcagagaaaacatgaaggaattaaaggaggccaggccgcgcaaagataacaggcgtcctgatctggaaatctataagcctggcctttctcggctaaggaacaagcccaaaatcaaggaaccccctgggagtgaggagttcaaagatgaaattgttaatgaccgagattgctctgctgttgaaaatggtacacagcccgttaaagatgtctgcaaggaactgaacaaccaagagcagaatggtcctatagacccggaaaataatcggggacaagaatcctttcctaggactgctggacaagaggatcgtagtctaaaaattatcaaaagaacaaagaaacccgacctgcagatctatcagcctggacgacgtttgcagactgttagcaaagaatccgccagtcgggtggaggaggaagaagtcctcaaccaggtagaacaactgagagtagaggaagatgagtgtaggggaaatgttgcgaaggaggaagttgcgaataaaccagacagggccgagatagaaaagagcccaggtggtgggagagtaggggctgcaaaaggagaaaaaggaaagaggatgggaaaaggggagggggtgagggaaacccacgacgacccggcccgcgggaggccgggctccgcaaagcgctactcccgctcagacaaacgaaggaatcgctaccgcacgcgcagcaccagctcagctggcagcaacaacagcgctgagggagctggcctgacggataatggatgtcgccgccgccgacaggataggaccaaggagaggccacgactgaagcagcaagtgtctgtgtcctcaaccgattccttagacgaggacagaattgatgagcctgatggattaggacccaggagaagttcagaaaggaagagacatttagaaagaaactggtctggccgtggggagggtgagcagaaaaccagtgctaaagaatatcgaggcactcttcgtgtcactttcgatgcagaagccatgaacaaagagtctcccatggtgaggtcagccagggatgatatggatagaggaaagcctgacaaaggcttgagcagtgggggcaaaggctctgagaagcaggagtccaaaaacccgaaacaagaacttcggggtcgtggtcgtggcattctgattttgcctgcccataccaccctatctgtcaattcagcaggttctccagagtccgcgcctttgggacctcggcttttgtttggatctggtagtaaggggtctcggagttggggccgtggaggcaccacacgccgattgtgggacccaaacaatcctgatcagaaacctgctctaaagactcatacgccccagctacatttcttggacactgatgatgaagtcagccctacatcttggggtgactcacgccaggctcaggcatcttactataagtttcaaaactctgacaacccctattattacccccggacaccaggccctgcctcccagtatccctatacgggctataaccctctacagtacccagtgggccctacgaatggtgtgtacccagggccttactacccaggctacccgactccgtcaggacagtatgtgtgtagccctctgcctaccagcaccatgagtcccgaggaggtagagcagcacatgaggaacctgcagcaacaggagctgcacaggcttctccgggtggctgacaaccaggaactgcagctcagcaacctgctctccagggaccgcatcagtccggagggcctggagaagatggcgcaactcagagctgaactgctgcagctatatgagcgctgtattctattagatattgagttctctgataatcagaatgtggatcagatcctgtggaagaatgctttctatcaggtgattgagaagttcaggcaacttgtcaaggatccgaatgttgagaacccagaacagattcggaacagacttttggagctcttggatgagggtagtgacttctttgatagtttgcttcagaagctgcaggttacttacaagttcaaactggaagactacatggatggtcttgccattcgcagcaagccattacgcaagacagtaaaatatgccttgatcagtgcccagcgatgcatgatatgccaaggagatattgctaggtaccgggagcaagccagtgatacagcgaattatgggaaagcacgcagttggtacctgaaggcccagcacattgctcccaagaatgggcgcccctataaccagttggctttgctggcagtgtatacgaggaggaagcttgacgctgtctattactatatgcgcagtttagctgccagcaaccctatcctgactgccaaggagagtctcatgagcttgtttgaagagaccaagcggaaggcagaacagatggaaaagaagcaacatgaggaatttgacctgagccctgaccagtggcggaaaggaaagaagtctactttccggcatgttggagatgacaccactcgcctggagatctggattcatccatcccatccacggtcttcccagggcactgagtctgggaaggattctgagcaagagaatgggctgggcagcctgagtcccagtgatctgaacaaaaggttcatcctcagttttctccatgcccatgggaagctgtttacccggattgggatggagacattccctgcagtggctgagaaggtcctcaaggagttccaggtgttactgcagcacagcccctctcccattggaagtacccgcatgctgcagcttatgaccatcaatatgtttgcagtacacaactcccagctgaaagactgcttctcggaggagtgccgctctgtgatccaggaacaagccgcagctctgggcttggccatgttttctctactggtccgccgctgcacctgcttacttaaggagtccgccaaagctcagctgtcctctcctgaggaccaggatgaccaagacgacatcaaggtgtcttcctttgtcccggacctgaaggagctgctccccagtgtcaaagtctggtcagattggatgctcggctacccggacacctggaatcctcctcccacatccctggatctgccctcgcatgttgctgtggatgtatggtcgacgctggctgatttctgtaacatactgactgcagtgaatcagtctgaggtgccactgtacaaggacccggatgatgacctcacccttcttatcctggaagaggatcggcttctctcgggctttgtccccttgctggctgcccctcaggacccctgctacgtggagaaaacctcggataaggttattgcagctgactgcaaaagggtcacagtgctgaagtattttctggaagccctttgtggacaagaagagcctctgctggcattcaagggtggaaagtatgtgtcagtggcacccgtcccagacaccatgggaaaggaaatgggaagccaagagggaacacgactggaagatgaggaggaggatgtggtgattgaagactttgaggaagattcagaggctgaaggcagcggaggcgaggatgacatcagggagcttcgggccaagaagctggctctggccaggaagatagctgagcagcagcgtcgccaggaaaagatccaggctgtcctggaggaccacagtcagatgaggcagatggagctcgaaatcagacctttgttcctcgtaccagacaccaacggcttcattgaccacctggccagtctggcgcggctgctggagagcaggaagtacatcctggtggtgcccctcatcgtgatcaatgagctggacggcctggccaaggggcaggagacagaccaccgggctgggggctacgcccgtgtggtacaagagaaggcccgcaagtccatcgagttcctcgagcagcgattcgagagtcgggactcttgcctgcgagccctgaccagccgtggcaatgaactcgaatccatcgccttccgcagtgaggacatcactggccagctgggtaacaacgatgatctcatcctgtcctgctgcctccactactgcaaagacaaggctaaggacttcatgcccgccagcaaagaggagccaatccggctactgcgggaggtggtgctgttgacggatgaccggaacctgcgtgtgaaggcgctcacaaggaatgttcctgtacgggacatcccagccttcctcacgtgggcccaggtgggctga

SEQ ID NO: 2 - protein, 1419 aa, Homo sapiens
MAEGLERVRISASELRGILATLAPQAGSRENMKELKEARPRKDNRRPDLEIYKPGLSRLRNKPKIKEPPGSEEFKDEIVNDRDCSAVENGTQPVKDVCKELNNQEQNGPIDPENNRGQESFPRTAGQEDRSLKIIKRTKKPDLQIYQPGRRLQTVSKESASRVEEEEVLNQVEQLRVEEDECRGNVAKEEVANKPDRAEIEKSPGGGRVGAAKGEKGKRMGKGEGVRETHDDPARGRPGSAKRYSRSDKRRNRYRTRSTSSAGSNNSAEGAGLTDNGCRRRRQDRTKERPRLKQQVSVSSTDSLDEDRIDEPDGLGPRRSSERKRHLERNWSGRGEGEQKTSAKEYRGTLRVTFDAEAMNKESPMVRSARDDMDRGKPDKGLSSGGKGSEKQESKNPKQELRGRGRGILILPAHTTLSVNSAGSPESAPLGPRLLFGSGSKGSRSWGRGGTTRRLWDPNNPDQKPALKTHTPQLHFLDTDDEVSPTSWGDSRQAQASYYKFQNSDNPYYYPRTPGPASQYPYTGYNPLQYPVGPTNGVYPGPYYPGYPTPSGQYVCSPLPTSTMSPEEVEQHMRNLQQQELHRLLRVADNQELQLSNLLSRDRISPEGLEKMAQLRAELLQLYERCILLDIEFSDNQNVDQILWKNAFYQVIEKFRQLVKDPNVENPEQIRNRLLELLDEGSDFFDSLLQKLQVTYKFKLEDYMDGLAIRSKPLRKTVKYALISAQRCMICQGDIARYREQASDTANYGKARSWYLKAQHIAPKNGRPYNQLALLAVYTRRKLDAVYYYMRSLAASNPILTAKESLMSLFEETKRKAEQMEKKQHEEFDLSPDQWRKGKKSTFRHVGDDTTRLEIWIHPSHPRSSQGTESGKDSEQENGLGSLSPSDLNKRFILSFLHAHGKLFTRIGMETFPAVAEKVLKEFQVLLQHSPSPIGSTRMLQLMTINMFAVHNSQLKDCFSEECRSVIQEQAAALGLAMFSLLVRRCTCLLKESAKAQLSSPEDQDDQDDIKVSSFVPDLKELLPSVKVWSDWMLGYPDTWNPPPTSLDLPSHVAVDVWSTLADFCNILTAVNQSEVPLYKDPDDDLTLLILEEDRLLSGFVPLLAAPQDPCYVEKTSDKVIAADCKRVTVLKYFLEALCGQEEPLLAFKGGKYVSVAPVPDTMGKEMGSQEGTRLEDEEEDVVIEDFEEDSEAEGSGGEDDIRELRAKKLALARKIAEQQRRQEKIQAVLEDHSQMRQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVG

SEQ ID NO: 3 - DNA, 3051 bp, Homo sapiens
  CDS (1)..(3051)
atgagccaaggcccccctacaggggagagcagcgagcccgaagcaaaagtcctccacactaagcggctttaccgggctgtggtggaggctgtgcatcgacttgacctcatcctttgcaacaaaactgcttatcaagaagtattcaaaccagaaaacattagcctgaggaacaagctgcgtgagctctgcgtcaagcttatgttcctgcacccagtggactatgggagaaaggctgaggagctgctgtggagaaaggtatactatgaagttatccagcttatcaagactaacaaaaagcacatccacagccggagcactttggaatgtgcctacaggacgcacctggttgctggtattggcttctaccagcatctccttctctatatccagtcccactaccagctggaactgcagtgctgcatcgactggacccatgtcactgaccccctcataggatgcaagaagccagtgtctgcctcagggaaggagatggattgggcacagatggcatgtcaccgatgtctggtgtatctgggggatttgtcccgatatcagaatgaattagctggcgtagataccgagctgctagccgagagattttactaccaagccctgtcagtagctcctcagattggaatgcccttcaatcagctgggcaccctggcaggcagcaagtactataatgtggaagccatgtattgctacctgcgctgcatccagtcagaagtgtcctttgagggagcctatgggaacctcaagcggctgtatgacaaggcagccaaaatgtaccaccaactgaagaagtgtgagactcggaaactgtctcctggcaaaaagcgatgtaaagacattaaaaggttgctagtgaactttatgtatctgcaaagcctcctacagcccaaaagcagctccgtggactcagagctgacctcactttgccagtcagtcctggaggacttcaacctctgcctcttctacctgccctcctcacccaacctcagcctggccagtgaggatgaggaggagtatgagagtggatatgctttcctcccggaccttctcatctttcaaatggtcatcatctgccttatgtgtgtgcacagcttggagagagcaggatccaagcagtacagtgcagccattgccttcaccctggccctcttttcccacctcgtcaatcatgtcaacatacggctgcaggctgagctggaagagggcgagaatcccgtcccggcattccagagtgatggcacagatgaaccagagtccaaggaacctgtggagaaagaggaggagccagatcctgagcctcctcctgtaacaccccaagtgggtgagggcagaaagagccgtaagttctctcgcctctcctgtctccgccgtcgccgccacccacccaaagttggtgatgacagtgacctgagtgaaggctttgaatcggactcaagccatgactcagcccgggccagtgagggctcagacagtggctctgacaagagtcttgaaggtgggggaacggcctttgatgctgaaacagactcggaaatgaatagccaggagtcccgatcagacttggaagatatggaggaagaggaggggacacggtcaccaaccctggagccccctcggggcagatcagaggctcccgattccctcaatggcccactgggccccagtgaggctagcattgccagcaatctacaagccatgtccacccagatgttccagactaagcgctgcttccgactggcccccacctttagcaacctgctcctccagcccaccaccaaccctcatacctcggccagccacaggccttgcgtcaatggggatgtagacaagccttcagagccagcctctgaggagggctctgagtcggaggggagtgagtccagtggacgctcctgtcggaatgagcgcagcatccaggagaagcttcaggtcctgatggccgaaggtctgcttcctgctgtgaaagtcttcctggactggcttcggaccaaccccgacctcatcatcgtgtgtgcacagagctctcaaagtctgtggaaccgcctgtctgtgttgctgaatctgttgcctgctgctggtgaactccaggagtctggcctggccttgtgtcctgaggtccaagatcttcttgaaggttgtgaactgcctgacctcccctctagccttctgctcccagaggacatggctcttcgtaacctgcccccgctccgagctgcccacagacgctttaactttgacacggatcggcccctgctcagcaccttagaggagtcagtggtgcgcatctgctgcatccgcagctttggtcatttcatcgcccgcctgcaaggcagcatcctgcagttcaacccagaggttggcatcttcgtcagcattgcccagtctgagcaggagagcctgctgcagcaggcccaggcacagttccgaatggcacaggaggaagctcgtcggaacaggctcatgagagacatggctcagctacgacttcagctcgaagtgtctcagctggagggcagcctgcagcagcccaaggcccagtcagccatgtctccctacctcgtccctgacacccaggccctctgccaccatctccctgtcatccgccaactggccaccagtggccgcttcattgtcatcatcccaaggacagtgatcgatggcctggatttgctgaagaaggaacacccaggggcccgggatgggattcggtacctggaggcagagtttaaaaaaggaaacaggtacattcgctgccagaaagaggtgggaaagagctttgagcggcataagctgaagaggcaggatgcagatgcctggactctctataagatcctagacagctgcaaacagctgactctggcccagggggcaggtgaggaggatccgagtggcatggtgaccatcatcacaggccttccactggacaaccccagcgtgctttcaggccccatgcaggcagccctgcaggccgctgcccacgccagtgtggacatcaagaatgttctggacttctacaagcagtggaaggaaattggttga

SEQ ID NO: 4 - protein, 1016 aa, Homo sapiens
MSQGPPTGESSEPEAKVLHTKRLYRAVVEAVHRLDLILCNKTAYQEVFKPENISLRNKLRELCVKLMFLHPVDYGRKAEELLWRKVYYEVIQLIKTNKKHIHSRSTLECAYRTHLVAGIGFYQHLLLYIQSHYQLELQCCIDWTHVTDPLIGCKKPVSASGKEMDWAQMACHRCLVYLGDLSRYQNELAGVDTELLAERFYYQALSVAPQIGMPFNQLGTLAGSKYYNVEAMYCYLRCIQSEVSFEGAYGNLKRLYDKAAKMYHQLKKCETRKLSPGKKRCKDIKRLLVNFMYLQSLLQPKSSSVDSELTSLCQSVLEDFNLCLFYLPSSPNLSLASEDEEEYESGYAFLPDLLIFQMVIICLMCVHSLERAGSKQYSAAIAFTLALFSHLVNHVNIRLQAELEEGENPVPAFQSDGTDEPESKEPVEKEEEPDPEPPPVTPQVGEGRKSRKFSRLSCLRRRRHPPKVGDDSDLSEGFESDSSHDSARASEGSDSGSDKSLEGGGTAFDAETDSEMNSQESRSDLEDMEEEEGTRSPTLEPPRGRSEAPDSLNGPLGPSEASIASNLQAMSTQMFQTKRCFRLAPTFSNLLLQPTTNPHTSASHRPCVNGDVDKPSEPASEEGSESEGSESSGRSCRNERSIQEKLQVLMAEGLLPAVKVFLDWLRTNPDLIIVCAQSSQSLWNRLSVLLNLLPAAGELQESGLALCPEVQDLLEGCELPDLPSSLLLPEDMALRNLPPLRAAHRRFNFDTDRPLLSTLEESVVRICCIRSFGHFIARLQGSILQFNPEVGIFVSIAQSEQESLLQQAQAQFRMAQEEARRNRLMRDMAQLRLQLEVSQLEGSLQQPKAQSAMSPYLVPDTQALCHHLPVIRQLATSGRFIVIIPRTVIDGLDLLKKEHPGARDGIRYLEAEFKKGNRYIRCQKEVGKSFERHKLKRQDADAWTLYKILDSCKQLTLAQGAGEEDPSGMVTIITGLPLDNPSVLSGPMQAALQAAAHASVDIKNVLDFYKQWKEIG

SEQ ID NO: 5 - protein, 1265 aa, Homo sapiens
  MOD_RES (1): Variable amino acid
XCLKPFYLMRNWMFLQIVIVGENVSFKSQMRTENLKSEEHLKSSNIRQAEVLKADMTDSKLGPAEVWTSRQALQDLYQKMLVTDLEYALDKKVEQDLWNHAFKNQITTLQGQAKNRANPNRSEVQANLSLFLEAASGFYTQLLQELCTVFNVDLPCRVKSSQLGIISNKQTHTSAIVKPQSSSCSYICQHCLVHLGDIARYRNQTSQAESYYRHAAQLVPSNGQPYNQLAILASSKGDHLTTIFYYCRSIAVKFPFPAASTNLQKALSKALESRDEVKTKWGVSDFIKAFIKFHGHVYLSKSLEKLSPLREKLEEQFKRLLFQKAFNSQQLVHVTVINLFQLHHLRDFSNETEQHTYSQDEQLCWTQLLALFMSFLGILCKCPLQNESQEESYNAYPLPAVKVSMDWLRLRPRVFQEAVVDERQYIWPWLISLLNSFHPHEEDLSSISGHQGITGDKEGQQRRIRQQRLISIGKWIADNQPRLIQCENEVGKLLFITEIPELILEDPSEAKENLILQETSVIESLAADGSPGLKSVLSTSRNLSNNCDTGEKPVVTFKENIKTREVNRDQGRSFPPKEVRRDYSKGITVTKNDGKKDNNKRKTETKKCTLEKLQETGKQNVAVQVKSQTELRKTPVSEARKTPVTQTPTQASNSQFIPIHHPGAFPPLPSRPGTFLQPTAHSPAGNQVQAGKQSHIPYSQQRPSGPGPMNQGPQQSQPPSQQPLTSLPAQPTAQSTSQLQVQALTQQQQSPTKAVPALGKSPPHHSGFQQYQQADASKQLWNPPQVQGPLGKIMPVKQPYYLQTQDPIKLFEPSLQPPVMQQQPLEKKMKPFPMEPYNHNPSEVKVPEFYWDSSYSMADNRSVMAQQANIDRRGKRSPGVFRPEQDPVPRMPFEDPKSSPLLPPDLLKSLAALEEEEELIFSNPPDLYPALLGPLASLPGRSLFERYPNNSMFNEVYGKNLTSSSKAELSPSMAPQETSLYSLFEGTPWSPSLPASSDHSTPASQSPHSSNPSSLPSSPPTHNHNSVPFSNFGPIGTPDNRDRRTADRWKTDKPAMGGFGIDYLSATSSSESSWHQASTPSGTWTGHGPSMEDSSAVLMESLKVSRFQEQEPQDYYSKVYLVQFHDASWTFCSGAAVNAAEAETATGTRHHEPSTLRPKWQPGNEGSINHGMLGLQDWPTQSPAGGSPLFCFSLSRGCKYSTSPLSVHEMFAVQQKEKSIRNSPSPRGPPEGEREELLFISLSYLVSPPLTFSIVHVPSHMGSES

SEQ ID NO: 6 - protein, 172 aa, Homo sapiens
MTCHHCLLYLRDLFCYQREILPLVTKNLAERCYYRALSVALHMGMPFNLLGAITGNKYHDIESMYFYQHWLYSEVPFKGASWSLKGLFDHAGKSASGIKYKDDMSSFFELEPPTGSYLGQNASIGQFHNTLQTIKKKLKILSVGGTAAHHQGDAWLALCQPQPPQLTLVEST

SEQ ID NO: 7 - protein, 40 aa, Homo sapiens
KDKAKDFMPASKEEPIRLLREVVLLTDDRNLRVKALTRNV

SEQ ID NO: 8 - protein, 40 aa, Homo sapiens
KSFERHKLKRQDADAWTLYKILDSCKQLTLAQGAGEEDPS

SEQ ID NO: 9 - protein, 207 aa, Saccharomyces cerevisiae
VIPLVLKLLWLQIHEPTLQWFEHWFHDIMRLSNRRKFRVFRIFQKKMIQFFKITHRYYYDIIEHICAKYDMNSVISNALFAKLNLMQYTDGLSTHEKIILNTSNPLTFSIVISLQRCVINLGSTHFYKTLLNKPSNKPKSVEGFEKSIRYLNIASLYLPAVGDTYFQRAKIYLITGKFSLYFFELVRGALVRIPSKCALNNLKDFIL

SEQ ID NO: 10 - protein, 213 aa, Saccharomyces cerevisiae
SISSILDFSWESVHYPIFKWFQMWRNYILFEKENKKQQTKFIDFRKMNSKMLKFFKTVQNFYVNVINTVYKKYDISVLLPKRIIQDLKLSDIENTTNVGDILAVKTFNSSSPLAHLIPTLFHRCLLFLGTAYRYKTLLEEISNKYSISNFKKSLDFFRLASLVLPSAGETYSQAGAIFLQTGNLGIAVFNFVKGMMTKMPSPVSIKNFGALMV

SEQ ID NO: 11 - protein, 192 aa, Schizosaccharomyces pombe
DIIWSCCHYKIIQHFRSRFREIHPRHVVEKKKTKKVFFKFLKTCAIFYQTCISELISKFQLDSYRPFFCKWTSSATVSSTISNDEMSSIPEASYSRNHMEALECVYNCFIYLGDMARYSSTCLKKRGAYDRALGFYDLAHRTLPGNGMHRNQIAVVWASDECIVESIYWFSSALCSEDPPKSALLNLLKQLI

SEQ ID NO: 12 - protein, 222 aa, Schizosaccharomyces pombe
MEKDVLTYLWMRVHYQVISFFKHRIYEASTQHDPELLSSLVTMHIQYLNSTIQFYTTLIAIIGELYHLQCLSPLTSFFTSCVTPKTILESPLRKQGSHNWKTSTNSQSRLAALFSSIFEDSCLEVDSVKRLLSGSPSSSSSPLKKDSSSNSLTYEPALTDHKPQYLVLCVYRSLIYIGDVHRYLAEVRSPNVPDYQVSRRYYVMAANVAPDYGVHFHQLGLI

SEQ ID NO: 13 - protein, 77 aa, Caenorhabditis elegans
LLRMGDLMRYKENYPKAQEYYEQSCRINPADGAVWNQLGLISSLGAKNLESVYFHTRALHATMEFPTASGGLTNIFK

SEQ ID NO: 14 - protein, 204 aa, Drosophila melanogaster
VGKKVREVMWRRGYYEFIAFVKKNWHKQCQDAATAQENMQKLERFLCAGIFNYKRLSARMEEIYDLDLKYLIDFSIISDGYISDMIGDTMESSHSGTHAVDKSLEAVSFALDTIHSSLLSLGDLHRYFLDFRIDSKLSISKEQVAKYYLEAFKLNPAIGMAQNQLGTLHYGQNHDLDSTYHYLYSLVCIIPFELSENNLNKLFA